(19) United States
(12) Patent Application Publication
(10) Pub. No.: US 2023/0241228 A1
(43) Pub. Date: Aug. 3, 2023

(54) Biocampatible and Biodegradable Anionic Hydrogel System

(71) Applicant: BOARD OF TRUSTEES OF THE UNIVERSITY OF ARKANSAS, Fayetteville, AR (US)

(72) Inventors: Jingyi Chen, Fayetteville, AR (US); Tengjiao Wang, Fayetteville, AR (US); Isabelle Niyonshuti, Fayetteville, AR (US); Suresh Kumar Thallapuranam, Fayetteville, AR (US); Ravi K. Gundampati, Fayetteville, AR (US); Shilpi Agrawal, Fayetteville, AR (US); Kyle P. Quinn, Fayetteville, AR (US); Jake D. Jones, Fayetteville, AR (US)

(73) Assignee: BOARD OF TRUSTEES OF THE UNIVERSITY OF ARKANSAS, Fayetteville, AR (US)

(21) Appl. No.: 18/298,348

(22) Filed: Apr. 10, 2023

Related U.S. Application Data
(63) Continuation-in-part of application No. 17/183,083, filed on Feb. 23, 2021.
Application No. 17/183,083 is a division of application No. 16/409,495, filed on May 10, 2019, now abandoned.
(60) Provisional application No. 62/670,578, filed on May 11, 2018.

(51) Int. Cl. A61K 47/58; A61K 47/69; A61K 9/00; C08F 283/06; A61K 38/18; A61P 17/02; A61K 9/06

(52) U.S. Cl. CPC A61K 47/58; A61K 47/6903; A61K 9/0019; C08F 283/065; A61K 9/0024; A61K 38/1825; A61P 17/02; A61K 9/06

(57) ABSTRACT

An anionic hydrogel for wound healing comprised of poly(oligoethylene glycol monoacrylate), acrylic, a neutral species and a wild-type fibroblast growth factor 1 (wtFGF1).

### RELATED APPLICATIONS

This application is a continuation in part of U.S. Ser. No. 17/183,083 filed on Feb. 23, 2021, which is a divisional of U.S. Ser. No. 16/409,495 filed on May 10, 2019, which claims priority to U.S. Provisional Application Ser. No. 62/670,578 filed on May 11, 2018, both of which are hereby incorporated in their entirety.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

Hydrogels are three-dimensional, cross-linked polymer networks that can retain a large amount of water. They allow biomolecules to be trapped in their porous structures for delivery in applications such as tissue engineering and wound healing. In general, hydrogels can be made from both natural and synthetic polymers. Although natural polymers are often biocompatible and biodegradable, hydrogels made of natural polymers are difficult to be chemically functionalized for sustained release and/or potentially immunogenic in the host body. In contrast, hydrogels made from synthetic polymers have several advantages including modifiable chemical properties, tunable mechanical properties, controllable porosity and transport properties. Poly(ethylene glycol) (PEG) is commonly used to form hydrogels due to its good biocompatibility and non-immunogenity; however, PEG hydrogels typically possess minimal or no intrinsic biological activity due to the antifouling nature of the PEG polymers.

PEG is also known for its biocompatibility, neutrality, and anti-biofouling properties that are desired for the delivery systems. For this reason, PEG-hydrogels have shown an important property in providing a controlled and sustained release of active therapeutics for important cellular functions. Furthermore, PEG has been used to modify non-biocompatible and non-biodegradable polymers to have biocompatibility and biodegradability desired in biomedical applications. Despite their excellent properties, PEG hydrogels suffer from weak mechanical properties which can potentially limit their applications. Studies have shown that PEG hydrogels copolymerized with other monomers could enhance their chemical, physical, and mechanical properties. For instance, poly(acrylic acid) (PAA) was incorporated in PEG to make a copolymer network of PEG-PAA hydrogels with improved mechanical properties. Furthermore, PAA introduces negative charges to the neutral PEG for enhancement of gel functionalities for protein delivery; however, the charge distribution is difficult to control and often forms pocket containing high density of negatives charges in the gel that binds to positively-charged protein too tight to prevent their release. Here we develop a method to introduce neutral species to space out the negative charges in the gel and demonstrate the enhancement of protein release.

### BRIEF SUMMARY OF THE INVENTION

In other embodiments, the present invention provides a method, system, approach and solution that provide biocompatible and biodegradable hydrogels for sustained delivery of biological therapeutic agents.

In other embodiments, the present invention provides a system, method, approach and solution that provide biocompatible and biodegradable hydrogels for regenerative medicine applications.

In other embodiments, the present invention provides a system, method, approach and solution that provide a method to synthesize biocompatible and biodegradable anionic hydrogels based on poly(acrylic acid)-co-poly(oligoethylene glycol monoacrylate) for protein delivery.

In other embodiments, the present invention provides a system, method, approach and solution that provide a synthesis that involves an aqueous free radical polymerization.

In other embodiments, the present invention provides a system, method, approach and solution that provide a synthesis that involves an aqueous free radical polymerization, followed by sequential steps to allow the swelling of copolymer into the hydrogels.

In other embodiments, the present invention provides a system, method, approach, and solution wherein the introduction of an anionic group-containing acrylic acid into the copolymers changes the thermal properties and viscosity of the hydrogels due to the alternation of intermolecular interactions in the polymer networks. In these embodiments, electrostatic interaction(s) between the anionic hydrogels and positively charged proteins render the hydrogels capable of a sustainable release of the proteins.

In other embodiments, the present invention provides a system, method, approach and solution that provide an ionizable component, acrylic acid (AA), into the PEG-based hydrogel to form a biocompatible and biodegradable, anionic hydrogel of poly(acrylic acid)-co-poly(oligoethylene glycol monoacrylate) (PAA-co-POEGA).

In other embodiments, the present invention provides a system, method, approach and solution that provide a synthesis that involve an aqueous free radical polymerization, followed by sequential steps to allow the swelling of copolymer into the hydrogels. The presence of AA provides a negative charge in the hydrogel at physiological pH that improves the retention of the entrapped, positively charged proteins through electrostatic interactions for sustained delivery. PAA mimics heparin which possesses antithrombin-activating properties and may promote anti-inflammatory processes.

In other embodiments, the present invention provides a system, method, approach and solution wherein monomers AA and PEG acrylate (OEGA) may be polymerized by a cross-linker such as N,N′-methylenebis(acrylamide) (MBAm) or N,N′-bis(acryloyl)cystamine (BAC). Depending on the ratio of AA/OEGA/cross-linker, the chemical and physical properties of the resulting hydrogel may be tuned to facilitate the protein delivery for different applications.

In other embodiments, the present invention provides a system, method, approach and solution wherein the sustained release of proteins from the hydrogel may be attained by using both the model protein lysozyme and wild-type fibroblast growth factor 1 (wtFGF1) that are positively charged under the physiological condition.

In other embodiments, the present invention provides a system, method, approach and solution wherein the injectable PAA-co-POEGA hydrogel is biocompatible. The biodegradability of the hydrogel may be attained by hydrogel cross-linking BAC.

In other embodiments, the present invention provides a method, system, approach and solution that provide biocompatible and biodegradable hydrogels for sustained delivery of biological therapeutic agents.

In other embodiments, the present invention provides a method, system, approach and solution that provide biocompatible and biodegradable hydrogels for regenerative medicine applications.

In other embodiments, the present invention provides a method, system, approach and solution that provide a method to synthesize biocompatible and biodegradable anionic hydrogels based on poly(acrylic acid)-co-poly(oligoethylene glycol monoacrylate) for protein delivery.

In other embodiments, the present invention provides a method, system, approach and solution that involves an aqueous free radical polymerization, followed by steps to allow the swelling of copolymer into the hydrogels.

In other embodiments, the present invention provides a method, system, approach and solution wherein the introduction of an anionic group-containing acrylic acid into the copolymers changes the thermal properties and viscosity of the hydrogels due to the alternation of intermolecular interactions in the polymer networks.

In other embodiments, the present invention provides a method, system, approach and wherein there is an electrostatic interaction(s) between the anionic hydrogels and positively charged proteins, thus, rendering the hydrogels capable of a sustainable release of the proteins.

In other embodiments, the present invention provides a method, system, approach and solution that provide an ionizable component, acrylic acid (AA), into the PEG-based hydrogel to form a biocompatible and biodegradable, anionic hydrogel of poly(acrylic acid)-co-poly(oligoethylene glycol monoacrylate) (PAA-co-POEGA).

In other embodiments, the present invention provides a method, system, approach and solution that involve an aqueous free radical polymerization, followed by sequential steps to allow the swelling of copolymer into the hydrogels.

In other embodiments, the present invention provides a method, system, approach and solution wherein the presence of AA provides a negative charge in the hydrogel at physiological pH that improves the retention of the entrapped, positively charged proteins through electrostatic interactions for sustained delivery.

In other embodiments, the present invention provides a method, system, approach and solution wherein PAA mimics heparin which possesses antithrombin-activating properties and may promote anti-inflammatory processes.

In other embodiments, the present invention provides a method, system, approach and solution wherein the hydrogel synthesis consists of a two-step procedure involving a free radical polymerization to form a copolymer network followed by allowing the copolymer network swelling into hydrogel.

In other embodiments, the present invention provides a method, system, approach and solution wherein monomers AA and PEG acrylate (OEGA) may be polymerized by a cross-linker such as N,N′-methylenebis(acrylamide) (MBAm) or N,N′-bis(acryloyl)cystamine (BAC).

In other embodiments, the present invention provides a method, system, approach and solution wherein, depending on the ratio of AA/OEGA/cross-linker, the chemical and physical properties of the resulting hydrogel may be tuned to facilitate the protein delivery for different applications.

In other embodiments, the present invention provides a method, system, approach and solution wherein the sustained release of proteins from the hydrogel may be attained by using both the model protein lysozyme and wild-type fibroblast growth factor 1 (wtFGF1) that are positively charged under the physiological condition.

In other embodiments, the present invention provides a method, system, approach and solution wherein the injectable PAA-co-POEGA hydrogel is biocompatible. The biodegradability of the hydrogel may be attained by hydrogel cross-linking BAC.

In other embodiments, the present invention provides a method, system, approach and solution wherein the synthesis of the injectable hydrogels consists of a two-step procedure, the initial step involves an aqueous free radical polymerization of AA and OEGA (M.W. 480) with a cross-linking agent such as MBAm or BAC and the second step is to let the PAA-co-POEGA polymeric network swell in buffered solution such as a saline solution with 0.1 wt. % Borax (antibacterial agent), forming the injectable hydrogels.

In other embodiments, the present invention provides a method, system, approach and solution wherein the reaction may be initiated by APS to form PAA-co-POEGA polymeric network.

In other embodiments, the present invention provides a fast and simple synthesis method for the preparation of anionic hydrogels based on poly(oligo ethylene glycol monoacrylate-co-acrylic acid) P(OEGA-co-AA) using a free polymerization reaction.

In other embodiments, the present invention provides a preparation of anionic hydrogels based on poly(oligo ethylene glycol monoacrylate-co-acrylic acid) P(OEGA-co-AA) for controlled release of positively charged proteins such as lysozyme.

In other embodiments, the present invention provides an anionic injectable hydrogel developed for protein delivery based on poly(oligoethylene glycol monoacrylate-co-acrylic acid-co-N-isopropylacrylamide) or P(OEGA-co-AA-co-NIPAM) that is synthesized via a free-radical polymerization reaction.

In other embodiments, the present invention provides an anionic injectable hydrogel developed for protein delivery based on poly(oligoethylene glycol monoacrylate-co-acrylic acid-co-N-isopropylacrylamide) or P(OEGA-co-AA-co-NIPAM) that is synthesized via a free-radical polymerization reaction and incorporating PNIPAM to the P(OEGA-co-AA) spaces out the charge distribution of the anionic gel allowing for the increased release of positively-charged proteins.

In other embodiments, rather than incorporating PNIPAM to the P(OEGA-co-AA), other neutral species such as gelatin, may be used.

For the embodiments of the present invention, a release study was carried out using human acidic fibroblast growth factor (hFGF1) under physiological conditions. The results indicated that the release kinetics from the P(OEGA-co-AA-co-NIPAM) gel is temperature-dependent and release rate was increased by six times than the P(OEGA-co-AA) gel due to weaker electrostatic interactions between hFGF1 and the P(OEGA-co-AA-co-NIPAM) gel. This charge-driven release mechanism was also evidenced by much faster and sustained release kinetics of negatively-charged proteins from the P(OEGA-co-AA-co-NIPAM) gel compared to that of hFGF1. The released hFGF1 remained bioactive and promoted fibroblast's proliferation in vitro and skin wound healing in vivo. This study demonstrates a delivery system that enables sustained release of bioactive positively charged proteins for enhanced wound healing.

In other embodiments, the present invention provides an additional component poly (N-isopropylacrylamide), PNIPAM, to the copolymer to tune the charge density of the P(OEGA-co-AA) anionic hydrogel to control the release of active hFGF1 for wound healing. PNIPAM is a charge-neutral polymer and copolymerizing with P(OEGA-co-AA) provides an avenue for adjusting the charge distribution of the anionic hydrogel thereby reducing the strength of electrostatic interactions to facilitate the release of hFGF1. Additionally, the hydrophobic (propyl group) and hydrophilic (amide group) moieties of the repeating unit render PNIPAM a thermo-responsive polymer known to have a lower critical solution temperature (LCST) close to human body temperature. PNIPAM-based hydrogels have been used as drug delivery systems as they can release drugs due to the change of their structural conformation at their LCST. These hydrophobic and hydrophilic moieties can provide additional interactions to regulate the protein control release of the anionic hydrogels.

In other embodiments, the present invention synthesized P(OEGA-co-AA-co-NIPAM) via a free radical polymerization and formulated the anionic hydrogels intending to improve the release of active hFGF1 for enhanced wound healing. Vibrational spectroscopy was used to monitor the formation of the copolymer P(OEGA-co-AA-co-NIPAM). The water content of the hydrogel and thermal stability of the copolymers were studied by thermogravimetric analysis (TGA). No cytotoxic effect of the hydrogels was observed for the copolymer P(OEGA-co-AA-co-NIPAM).

In other embodiments, the present invention synthesized an injectable anionic biocompatible hydrogel system based on P(OEGA-co-AA-co-NIPAM) using a simple radical polymerization reaction. AA was added to the hydrogel to introduce negative charges in the hydrogel and NIPAM was used to space out the negative charges within the hydrogel to control the release rate of positively charged proteins. FTIR and Raman spectroscopy verified the chemical structure of the copolymers. DSC and TGA data shows increasing molar percentage of AA and NIPAM in the hydrogel increases thermal stability of the hydrogel. It was also observed that more NIPAM in the hydrogel cause the hydrogel to lose its water much faster and have a greater weight loss. P(OEGA-co-AA-co-NIPAM) hydrogel showed the capacity for a sustained release of hFGF1 with preserved bioactivity in physiological conditions. The sustained release was based on electrostatic interactions that held the proteins in the hydrogel and sustained its bioactivity overtime. Incorporating NIPAM in P(OEGA-co-AA) hydrogel system increased release rate of hFGF1 which may be explained by the neutral PNIPAM spacing out and/or lessening of negative charges on the anionic hydrogel, thus, reducing the interactions between the anionic hydrogels and the positively charged proteins. PNIPAM thermo-responsive properties also enhanced release profile at 37° C. compared to 25° C. because of the change in hydrogel structure to repel loaded protein upon increase in temperature above PNIPAM's LCST. Most of the encapsulated BSA was completely released from P(OEGA-co-AA-co-NIPAM) hydrogel because of its negative net charges that repel with similarly charged hydrogel and led to much faster release rate than hFGF1. The release profile of BSA confirm the importance of electrostatic interactions between the hydrogel and the protein resulting in a sustained release of the protein.

In other embodiments of the present invention, hFGF1 remains active after encapsulation, that the rate of sustained delivery of FGFs can be tuned by hydrogel synthesis, and that the hydrogel can be used as a delivery vehicle for positively charged proteins such as FGFs in wound healing applications.

In another embodiment, the present invention provides a method of producing an anionic hydrogel comprising the steps of: providing a) poly(oligoethylene glycol monoacrylate), acrylic acid and N,N′-methylenebis(acrylamide), and water in a container bubbled with a gas before sealing the container; b) incubating; c) adding an initiator; d) performing one or more evacuate-refill cycles; e) terminating the polymerization by cooling; f) dialyzing against water using dialysis membrane; g) lyophilizing into a form of dry gel; and h) adding a wild-type fibroblast growth factor 1 (wtFGF1) and swelling the gel to form an anionic hydrogel. The method may further include the step of adding a neutral species to the poly(oligoethylene glycol monoacrylate) and the acrylic acid, the neutral species spaces out the charge distribution of the anionic hydrogel allowing for the increased release of the a wild-type fibroblast growth factor 1 (wtFGF1).

In another embodiment of the present invention, the neutral species may be NIPAM.

In another embodiment of the present invention, the weight ratio of the anionic hydrogel is poly(oligoethylene glycol monoacrylate) 850, acrylic acid 25, NIPAM 125.

In another embodiment of the present invention, the neutral species is gelatin.

In another embodiment of the present invention, the weight ratio of the anionic hydrogel is poly(oligoethylene glycol monoacrylate) 850, acrylic acid 25, gelatin 160.

In another embodiment, the present invention provides an anionic hydrogel for wound healing comprising: poly(oligoethylene glycol monoacrylate), acrylic, a neutral species and a wild-type fibroblast growth factor 1 (wtFGF1).

In another embodiment, the present invention provides a method of to enhance cell growth and cell adhesion to treat a wound in a mammal comprising the steps: applying an anionic hydrogel to the wound; the anionic hydrogel comprising: poly(oligoethylene glycol monoacrylate), acrylic, a neutral species and a wild-type fibroblast growth factor 1 (wtFGF1); and the hydrogel configured to controllably release the a wild-type fibroblast growth factor 1 (wtFGF1).

In another embodiment of the present invention, the hydrogel inhibits the degradation of the wild-type fibroblast growth factor 1 (wtFGF1).

In another embodiment, the present invention further includes the step of increasing the neutral species to the poly(oligoethylene glycol monoacrylate) and the acrylic acid, to increase the release of the a wild-type fibroblast growth factor 1 (wtFGF1).

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 shows the synthesis of the injectable hydrogels based on the copolymer networks of PAA-co-POEGA for an embodiment of the present invention.

FIG. 2 is a plot of the release curve of wtFGF1 and shaped hydrogel of the embodiment of the present invention.

FIG. 3 shows the lysozyme released profile as a function of time for several embodiments of the present invention.

FIG. 4 shows the loading capacity of lysozyme in the PAA-co-POEGA for various embodiments of the present invention at various AA concentrations (0, 0.5, 1, 1.5, 2 M) in DPBS (pH 7.4) at room temperature for 3 days.

FIG. 5A illustrates a loading and release study of the disk hydrogel of PAA-co-POEGA (1:1) with loading capacity of wtFGF1 as a function of time.

FIG. 5B illustrates a loading and release study of the disk hydrogel of PAA-co-POEGA (1:1) with wtFGF1 released from the hydrogel disk under different temperatures in DPBS (pH 7.4).

FIG. 6 is an in vivo biocompatibility study using a mouse model for GEL900 (left), 3M Telladerm Gel (middle), and no treatment (right) with the wound size being monitored for 10 days.

FIG. 7 illustrates the released wtFGF1 as a function of time for GEL900 and GEL800.

FIG. 8A illustrates a cell proliferation assay of the released wtFGF1 from GEL900 and GEL800 with no wtFGF1 as a control with cell counts of 3T3 fibroblast cells from day 0 to day 3.

FIG. 8B illustrates a cell proliferation assay of the released wtFGF1 from GEL900 and GEL800 with calculated active released wtFGF1 as a function of time.

FIG. 9A illustrates the synthesis of the Poly(acrylic acid)-co-poly(oligoethylene glycolmonoacrylate)-co-poly(N-isopropyl acrylamide) (PAA-co-POEGA-co-PNIPAM). The reaction involves an aqueous free radical polymerization of AA, OEGA (M.W. 480), and NIPAM with the cross-linking agent MBAm initiated by APS.

FIG. 9B shows when after the polymer network is formed, the PAA-co-POEGA-co-PNIPAM polymer will be swollen in buffered saline solution containing 0.1 wt. % Borax (antibacterial agent), forming the injectable hydrogels.

FIG. 10 shows the controlled release of wtFGF-1 from different formulations of injectable anionic PAA-co-POEGA-co-PNIPAM hydrogels with the release was done in physiological conditions at pH 7.4.

FIG. 11A is a schematic illustration of the reaction scheme for the synthesis of P(OEGA-co-AA-co-NIPAM) copolymer via a free-radical polymerization reaction initiated by APS for an embodiment of the present invention. The copolymer network is cross-linked by MBAm. The lyophilized copolymer powder is swelled in PBS aqueous to form hydrogel.

FIG. 11B shows the gels of the embodiments of the present invention indicating the difference in viscosity.

FIG. 12A is the FTIR absorbance spectra of P(OEGA-co-AA-co-NIPAM) copolymers of 8 different gel formulations.

FIG. 12B is the FTIR absorbance spectra of Gel 8 showing main representative peaks.

FIG. 12C is the FTIR absorbance spectra of ester/carbonate C═O peak intensity at 1730 cm⁻¹ normalized to that of the C—O at 1095 cm⁻¹.

FIG. 12D is the FTIR absorbance spectra of the C═O peak intensity at 1645 cm⁻¹ for amide normalized to ester/carbonate C═O peak intensity at 1730 cm⁻¹.

FIG. 13A provides DSC thermograms of the copolymers in the temperature range from 50 to 500° C.

FIG. 13B is zoomed in for 380-460° C. area.

FIG. 13C provides TGA curves of the hydrogel formulations in 0-500° C. temperature range.

FIG. 13D provides derivative plots of TGA curves with zoomed in to 300-500° C. area.

FIG. 14 provides MTT-assay viability data showing polymer concentration effect on cell viability after treatment with Gel 900 (P(OEGA-co-AA) and Gel 6 P(OEGA-co-AA-co-NIPAM).

FIG. 15A provides release profiles of hFGF1 as a function of time using fluorescence spectroscopy (λₑₓ=280 nm)) from Gel 8 at 25° C. and 37° C. and from Gel 900 at 37° C.

FIG. 15B shows the controlled release of BSA from Gel 8 (λₑₓ=295 nm) for 5 days at 37° C.

FIG. 15C provides bioactivity of released hFGF1 from Gel 8 at 37° C.

FIG. 15D shows the stability of hFGF1 in the cell culture media at 37° C. Data in A-C were measured in triplicate (n=3). Error bars represent standard deviation.

FIG. 16 provides an in vivo study showing normalized wound sizes for wound treated with just gel 8 as a control and Gel 8 loaded with hFGF1 across the study for 10 days.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

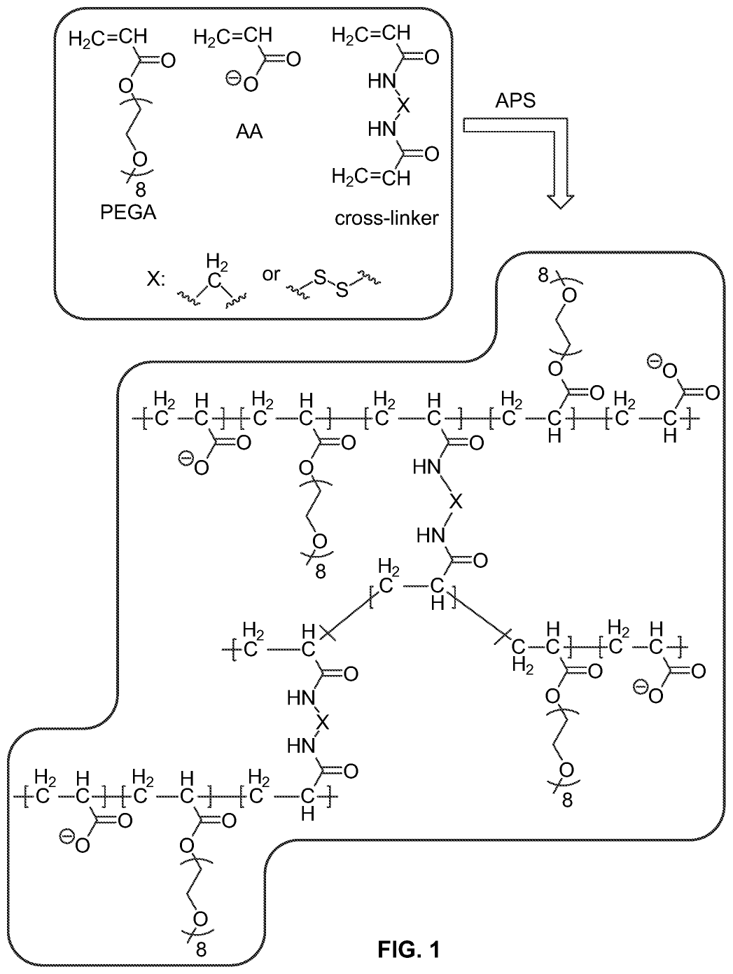

In other embodiments, the present involves the synthesis of the injectable hydrogels that consists of a two-step procedure (FIG. 1). The initial step involves an aqueous free radical polymerization of AA and OEGA (M.W. 480) with a cross-linking agent such as MBAm or BAC. The reaction was initiated by APS to form PAA-co-POEGA polymeric network. The second step is to let the PAA-co-POEGA polymeric network swell in a buffered solution such as a saline solution with 0.1 wt. % Borax (antibacterial agent), forming the injectable hydrogels. Five formulations were synthesized as listed in Table 1. It was determined that the fluidity decreased with increased AA component in the polymer chain. The chain length of the copolymer increased with increased weight percent of AA in the polymerization reaction. The longer the polymer chain, the stronger were the intermolecular interactions between the polymer chains leading to the distinctive difference in physical properties of the resulting hydrogels.

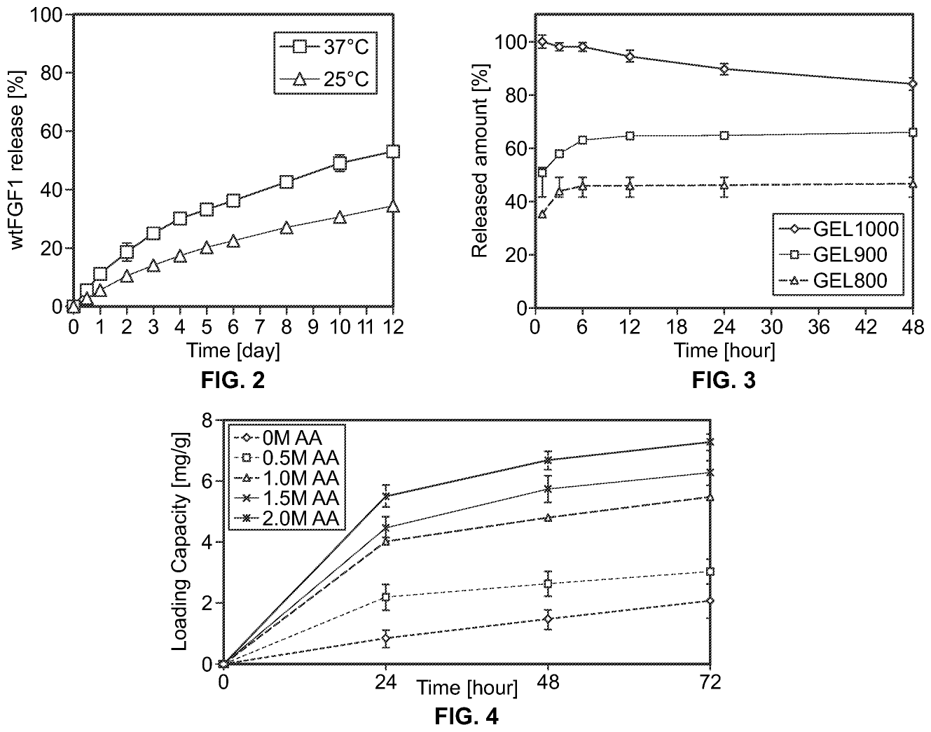

By increasing the concentrations of cross-linker and monomers, the hydrogels gained mechanical strength and became less fluidic. This property of hydrogel helped shaping of the hydrogel as pads or disks as shown in FIG. 2. The pad or disk hydrogel formulation is listed in Table 2.

TABLE 1

| | | | | | APS | | | | Borax |
|---|---|---|---|---|---|---|---|---|---|
| | | POEGA/AA | Mon./MBAm | MBAm | cont. | Reaction | Reaction | Total | content |
| | POEGA/AA | (mol/mol, | (mol/mol, | cont. | (w/v, | Temp | Time | Volume | (w/v, |
| Sample | (w/w, mg) | mmol) | mmol) | (mM) | %) | (° C.) | (min) | (mL) | %) |
| GEL1000 | 1000/0 | 2.08/0.00 | 2.08/0.1 | 10 | 1.5 | 70 | 30 | 10 | 0.1 |
| GEL950 | 950/50 | 1.98/0.69 | 2.67/0.1 | 10 | 1.5 | 70 | 15 | 10 | 0.1 |
| GEL900 | 900/100 | 1.88/1.39 | 3.26/0.1 | 10 | 1.5 | 70 | 5 | 10 | 0.1 |
| GEL850 | 850/150 | 1.77/2.08 | 3.85/0.1 | 10 | 1.5 | 70 | 5 | 10 | 0.1 |
| GEL800 | 800/200 | 1.67/2.78 | 4.44/0.1 | 10 | 1.5 | 70 | 5 | 10 | 0.1 |

TABLE 2

| C_{POEGA} (M) | C_{AA} (M) | C_{bis} (mM) | V (mL) | C_{APS} (mM) | T (° C.) |
|---|---|---|---|---|---|
| 0.5 | 0 | 50 | 1 | 50 | 70 |
| 0.5 | 0.5 | 50 | 1 | 50 | 70 |
| 0.5 | 1 | 50 | 1 | 50 | 70 |
| 0.5 | 1.5 | 50 | 1 | 50 | 70 |
| 0.5 | 2 | 50 | 1 | 50 | 70 |

For the injectable hydrogels, a model protein, lysozyme, was mixed with the hydrogels, to demonstrate the withholding capability of the hydrogels. The hydrogel-protein mixtures were placed in PBS and the released amount of lysozyme was monitored by fluorescence spectroscopy up to 48 hours (FIG. 3). Within the first hour, lysozyme was completely released from GEL1000, made from pure POEGA, while only 50% and 35% of lysozyme was released from GEL900 and GEL800, respectively. After 12 hours, the release reached equilibrium wherein 40% and 55% of lysozyme remained in GEL900 and GEL800, respectively suggesting a potential for sustainable release if placed on the wound site. As lysozyme is positively charged at physiological conditions, the increased concentration of AA in the copolymer will enhance the electrostatic interaction(s) between the polymeric network and positively charged proteins and consequently facilitates retention in the hydrogel for prolonged periods of time.

For the disk hydrogel, the loading capacity was assessed using the positively charged lysozyme (FIG. 4). The amount of lysozyme loaded in the hydrogels increased with increased AA concentration in the copolymer is the result of the electrostatic interaction(s) between negatively charged AA and lysozyme.

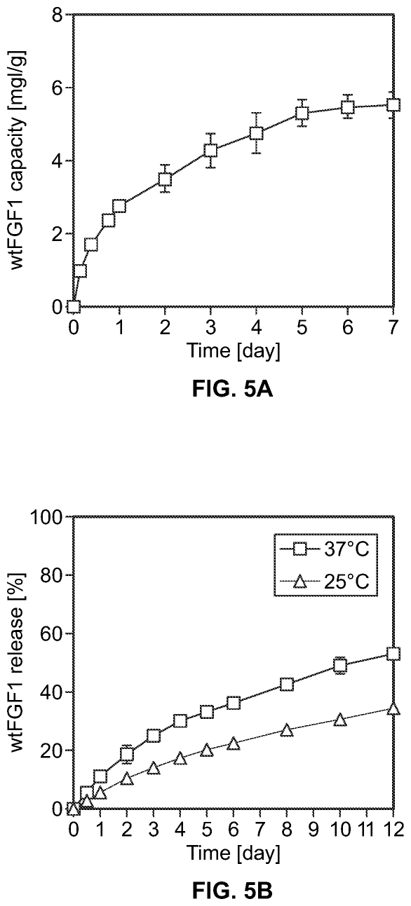

The wtFGF1 was further used to demonstrate the loading and releasing capacity of the disk hydrogel at a 1:1 molar ratio of AA/OEGA (FIG. 5). The loading capacity of disk hydrogels can reach ˜6 mg/g within a week similar to that of lysozyme. The release of wtFGF1 was very slow and temperature dependent. Over the course of 12 days, ˜50% and ˜30% wtFGF1 was released from the hydrogel at 37° C. and 25° C., respectively.

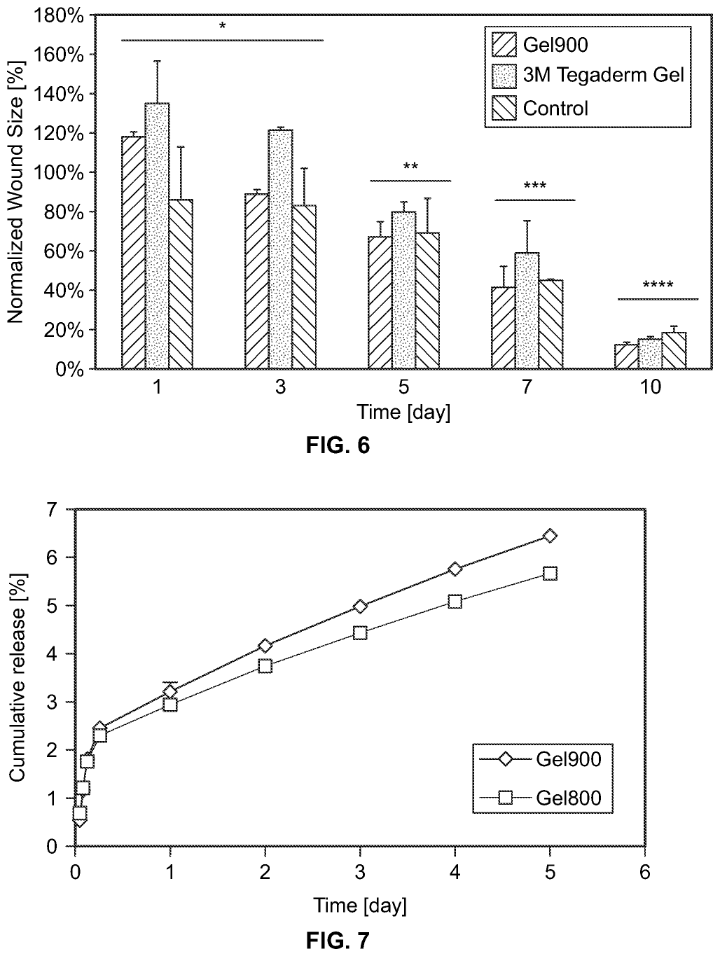

Since the viscosity of GEL900 is comparable to that of the commercially available 3M Tegaderm injectable hydrogel, it was further considered as a candidate for in vivo studies. The biocompatibility of GEL900 was evaluated using a mouse model of excisional wound healing. FIG. 6 shows the wound size comparison after individual application of GEL900 and the commercially available 3M Tegaderm Gel. On day 1, wounds treated with the GEL900 and 3M Tegaderm Gel increased in size by 20 to 30%. By day 3, the wounds applied with GEL900 began to decrease in size relative to the 3M Tegaderm Gel. By day 10, wounds treated with GEL900 had nearly closed, indicating biocompatibility. Similar to the control group without gel treatment, significant differences were observed between the day 1 or day 3 and the later days (i.e., day 5, 7, or 10) for both groups treated with gels. GEL900 appeared to be comparable to the 3M Tegaderm Gel during healing and neither of the gels was observed to delay the normal healing process.

In other embodiments, the present invention provides anionic hydrogels based on poly(acrylic acid)-co-poly(oligoethylene glycol monoacrylate) (PAA-co-POEGA) for protein delivery. The hydrogel comprises an injectable hydrogel prepared in two steps, one-pot method which involves an aqueous free radical polymerization, followed by sequential steps to allow the swelling of copolymer into the hydrogels. The hydrogel may then be retained in a number of different predetermined shapes such as a disk, cylinder, sphere and other shapes known to those of skill in the art.

A disk shape, redox-induced degradable hydrogel may also be prepared from the same monomer but crosslinked by redox-responsive cross-linking reagent. In a preferred embodiment, the injectable hydrogel comprises a copolymer comprising poly(oligoethylene glycol monoacrylate), acrylic acid, N,N′-methylenebis(acrylamide), sodium tetraborate and phosphate-buffered saline wherein a mass percentage of the components comprises: poly(oligoethylene glycol monoacrylate) ranging from 8%-10%; acrylic acid ranging from 2%-0%; N,N′-methylenebis(acrylamide) was fixed to 0.154%; sodium tetraborate was fixed to 0.1%; and phosphate-buffered saline up to 100%. The injectable hydrogel may also have a molecular weight of poly(oligoethylene glycol monoacrylate) of 480 g/mol.

In other aspects, the present invention provides a method of producing an injectable hydrogel comprising the steps of providing a) Poly(oligoethylene glycol monoacrylate), acrylic acid and N,N′-methylenebis(acrylamide) and water in a Schleck tube or other suitable container which may be bubbled with N₂ gas for 30 min before sealing the container which is followed by one or more and preferably three evacuate-refill cycles with Ar to remove the dissolved oxygen. The reaction solution may then be incubated at 70° C. for 30 min. After incubation, an initiator may be added under Ar flow, followed by another three evacuate-refill cycles with Ar. The reaction was allowed to proceed at 70° C. for 5-30 min under magnetic stirring at a speed of 350 rpm. Immediately after the reaction, the polymerization was terminated by cooling the container. The reaction solution was then dialyzed against water using dialysis membrane. The resulted gel solution was lyophilized into a form of a dry gel. The lyophilized copolymer was swollen PBS containing borax for 24 h to form the injectable anionic hydrogels.

In other steps, the polymerization may be initiated by ammonium persulphate and the molecular weight cut off of the dialysis membrane is 2 kDa. The viscosity of the injectable hydrogel may be controlled by the molar ratio between the total monomer and crosslink agent. The thermal stability of the injectable hydrogel may be controlled by the molar ratio between the total monomer and crosslink agent. In addition, one or more positively charged proteins may also be bonded to the injectable hydrogel by electrostatic interaction and the protein's capacity controlled by the molar concentration of acrylic acid. The positively charged protein release kinetic of the injectable hydrogel may be controlled by the molar concentration of acrylic acid.

In other aspects, the present invention provides a disk shape hydrogel comprising a copolymer comprising poly(oligoethylene glycol monoacrylate), acrylic acid, N,N′-methylenebis(acrylamide) and phosphate-buffered saline; wherein a mass percentage of the components may be as follows: poly(oligoethylene glycol monoacrylate) fixed to 24%; acrylic acid ranging from 0%-14.4%; N,N′-methylenebis(acrylamide) fixed to 0.77%; and phosphate-buffered saline up to 100%. The molecular weight of poly(oligoethylene glycol monoacrylate) may be 480 g/mol.

In other aspects, the present invention provides a method of producing cylindrical shaped hydrogel comprises of the following steps: providing poly(oligoethylene glycol monoacrylate), acrylic acid and N,N′-methylenebis(acrylamide) and water in a container which is bubbled with N₂ gas for 30 min before sealing the container and then adding an imitator. The reaction may be started by heating up to 70° C. for 4 h. The resulting hydrogel is washed preferably with water and allowed to completely swell in PBS for example. When the hydrogel is cylindrical in shape, the swelling ratio may be controlled by the molar ratio between the total monomer and crosslink agent. The polymerization may be initiated by ammonium persulphate.

A positively charged protein may be bonded to the shaped hydrogel by electrostatic interaction. The protein capacity may be controlled by the molar concentration of acrylic acid and the positively charged protein release kinetic of the disk shape hydrogel may be controlled by a competition between total protein capacity of hydrogel and molar concentration of acrylic acid.

In other aspects, the present invention provides a biodegradable disk shape hydrogel comprising a copolymer comprising poly(oligoethylene glycol monoacrylate), acrylic acid, N,N′-bis(acryloyl)cystamine and phosphate-buffered saline. The mass percentage of the components is as follows: poly(oligoethylene glycol monoacrylate) fixed to 24%; acrylic acid ranging from 3.6%-14.4%; N,N′-Methylenebis(acrylamide) fixed to 1.3%; and phosphate-buffered saline up to 100%. The molecular weight of poly(oligoethylene glycol monoacrylate) may be 480 g/mol.

In other aspects, the present invention provides a method of producing a disk shape hydrogel comprising the steps of: poly(oligoethylene glycol monoacrylate), acrylic acid, N,N′-bis(acryloyl)cystamine and water/EtOH mixture in a container was bubbled with N₂ gas for 30 min before sealing the container and adding an imitator with the reaction being started by heating up to 70° C. for 4 h. Next the resulting hydrogels are washed and allowed to swell to completion. In other embodiments, the polymerization may be initiated by ammonium persulphate and the volume ratio of water and EtOH is 3/1. The swelling ratio of the biodegradable disk shape hydrogel can be controlled by the molar ratio between the total monomer and crosslink agent and the positively charged protein may be bonded to the biodegradable disk shape hydrogel.

Other aspects of controlling aspects of the hydrogel include the following: the protein capacity may be controlled by the molar concentration of acrylic acid; the positively charged protein release kinetic of the disk shape hydrogel may be controlled by a competition between total protein capacity of hydrogel and the molar concentration of acrylic acid; and the biodegrade speed of the hydrogel may be controlled by the free thiol concentration.

Controlled Release of wtFGF1 from Injectable Anionic Hydrogels.

In another embodiment, 200 mg of injectable gel was mixed with 100 μL of 3.0 mg/mL wtFGF1 and stored at 4° C. overnight. The mixture was loaded into a transwell membrane plates insert (3 μm pore size) with a polyester membrane and polystyrene plates. The insert was then immersed in 1.5 mL of 1×PBS releasing media for controlled release at room temperature. At desired time points, 1 mL of the released medium was sampled and replaced by an equal volume of fresh medium to maintain a constant volume. The medium was then analyzed using fluorescence spectrophotometer. Then cumulative of released wtFGF1 was calculated from wtFGF1 calibration curve at 309 nm emission wavelength. The process was repeated three times and the results were reported as average values with standard deviations. The concentration of the FGF in each transwell is determined according to the measured fluorescence intensity using a standard curve. Unlike the lysozyme, the results in FIG. 7 indicate that there is little release of the wtFGF1 from the gels. This is possibly due to the strong electrostatic interaction of the wtFGF1 with the PAA component in the gel.

Cell Proliferation Assay for Bioactivity of Released wtFGF1.

Bioactivity of FGF was evaluated after release activity from PAA-co-POEGA hydrogels to ensure sustained activity. 3T3 fibroblast cells were grown to 80-90% confluency. Cell proliferation activity of released wtFGF1 was performed by incubating 10,000 cell/well with 50 ng/mL of released wtFGF1 in serum-supplemented medium. Cell proliferation assay was determined by CellTilter-Glo luminescent cell viability assay.

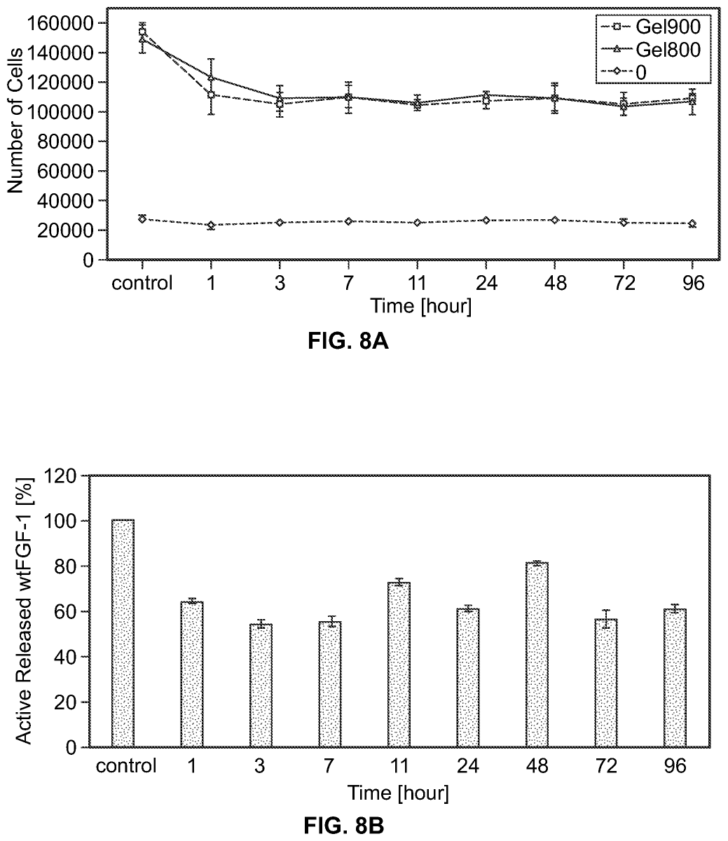

FIGS. 8A and 8B show the cell proliferation assay of the released wt.FGF1 from GEL900 and GEL800, respectively. Compared to the same amount of freshly prepared wtFGF1, the data shows about 60% of released wtFGF1 was active.

Poly(acrylic acid)-co-poly(oligoethylene glycolmonoacrylate)-co-poly(N-isopropyl acrylamide) Injectable Anionic Hydrogels for Controlled Release of wtFGF1 release.

Synthesis of PAA-co-POEGA-co-PNIPAM Injectable Hydrogels.

Each monomer was purified prior to the polymerization using the following methods. Oligoethylene glycol monoacrylate (Mₙ=480, OEGA) was purified through a basic Al₂O₃ column. Acrylic acid (AA) was purified through a neutral Al₂O₃ column. N-isopropyl acrylamide (NIPAM) was recrystallized from hexane. The polymer was synthesized by copolymerization of the three monomers, AA, OEGA, and NIPAM in an aqueous solution initiated by ammonium persulfate (APS). During the free radical polymerization, the monomers were cross-linked by N,N′-methylenebis (acrylamide) (MBAm). Typically, 10 mL aqueous solution containing a total amount of 1000 mg of OEGA, PAA and PNIPAM, and 15.4 mg MBAm (10 mM) was added to a 50-mL Schleck tube flask. The weight ratios of the three monomers in different formulation are listed in Table 3. The flask was bubbled with N₂ gas for 30 min before it was sealed. The flask was then subjected to three evacuate-refill cycles with Argon (Ar) to eliminate the dissolved oxygen. The reaction was incubated at 70° C. for 30 min. After incubation, 200 μL aqueous solutions of APS (15 mg) were added to the tube under Ar flow, followed by another three evacuate-refill cycles with Ar. The reaction could proceed at 70° C. for 5 min under magnetic stirring at 350 rpm. Immediately after the reaction, the polymerization was terminated by cooling the tube in an ice-water bath.

TABLE 3
Different formulations of PAA-co-POEGA-
co-PNIPAM injectable hydrogels.

| | Total | | | | |
|---|---|---|---|---|---|
| Sample | Volume | APS | MBAM | OEGA/AA/NIPAM | Temperature |
| ID | (mL) | (mg) | (mM) | w/w/w, mg | (° C.) |
| GEL 1 | 10 | 15 | 10 | 700/200/100 | 70 |
| GEL 2 | 10 | 15 | 10 | 900/50/50 | 70 |
| GEL 3 | 10 | 15 | 10 | 900/75/25 | 70 |
| GEL 4 | 10 | 15 | 10 | 800/150/50 | 70 |
| GEL 5 | 10 | 15 | 10 | 800/100/100 | 70 |
| GEL 6 | 10 | 15 | 10 | 800/50/150 | 70 |

PAA-co-POEGA-co-PNIPAM Gel.

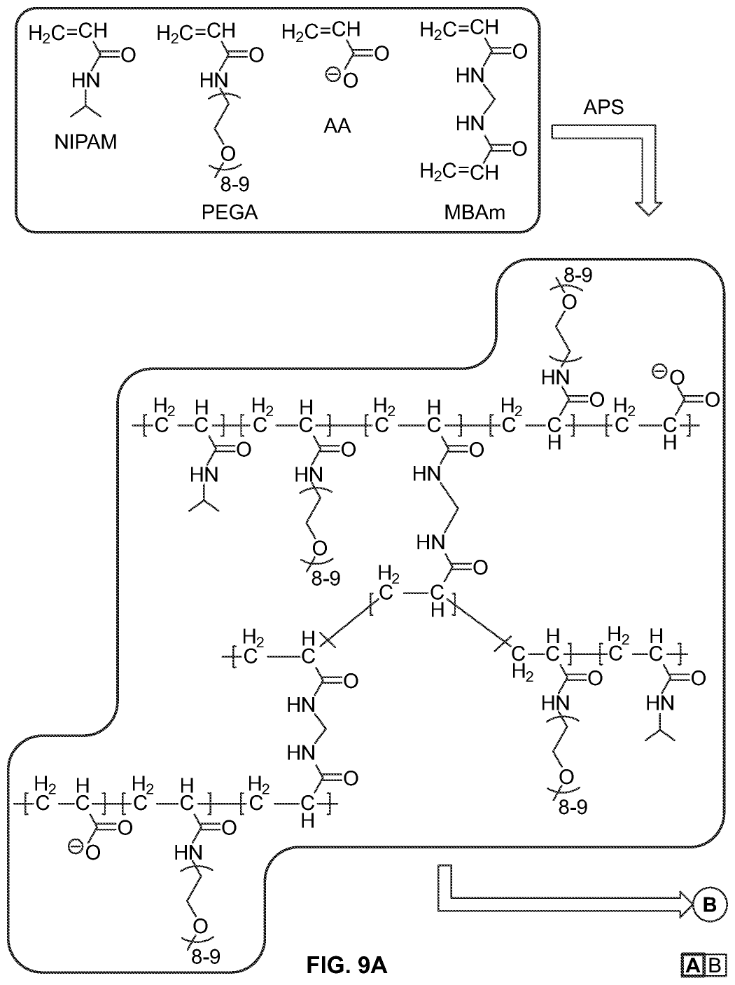

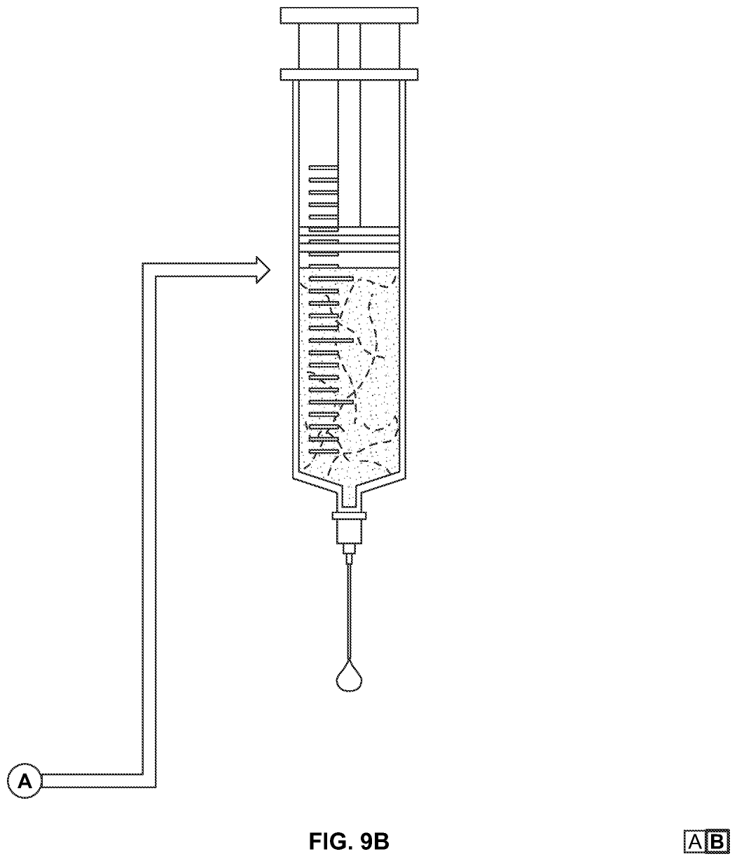

The newly developed anionic injectable hydrogels were prepared from the copolymer PAA-co-POEGA-co-PNIPAM, as shown in FIG. 9. The incorporation of the NIPAM in the polymer network may space out the negative charges in the two-component PAA-co-POEGA polymer. This may facilitate the release kinetics of wtFGF1 while maintaining the viscosity of the injectable gels for wound healing applications. In addition, the PNIPAM can response to heat changes that may be used for other applications that would involve with the use of heat as a tuning knob.

wtFGF1 Control Release Study.

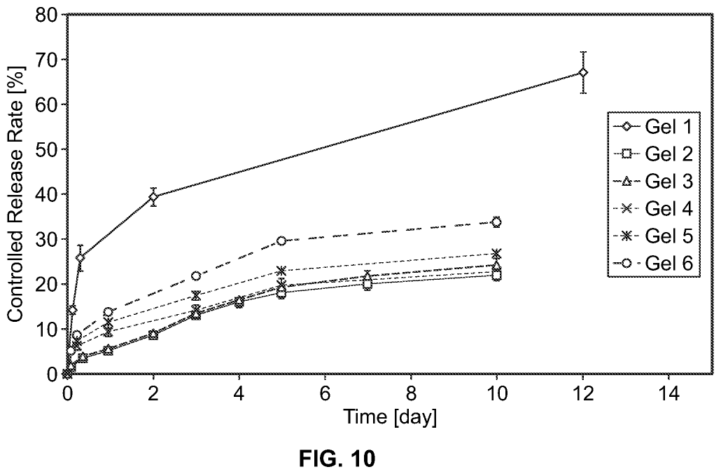

The release of wtFGF1 was significantly increased using PAA-co-POEGA-co-PNIPAM gels compared to PAA-co-POEGA, as shown in FIG. 10. The decrease of OEGA percentage in the formulation significantly increase the release kinetics of wtFGF1 from the gel. The release of wt-FGF1 could reach nearly 100% at 25 days (data not shown here). In addition, the decrease of the ratio AA/PNIPAM will increase the release kinetics of wtFGF1 from the gel providing a fine modulation of the wtFGF1 release.

Experimental Methods

Materials and Chemicals.

Oligo ethylene glycol monoacrylate (OEGA, Mₙ=480), Acrylic acid (AA), Dulbecco's Phosphate Buffered Saline (DPBS, 10 mM, pH 7.4) were purchased from Sigma-Aldrich. N-Isopropylacrylamide (NIPAM) was purchased from TCI. N,N′Methylenebisacrylamide (MBAm) and Potassium bromide (KBr) were purchased from Alfa Aesar. Ammonium persulfate (APS) was purchased from VWR. Dialysis membrane (MWCO 2 kDa) was purchased from Spectrum Laboratories. Transwell membrane plates were purchased from Corning.

Inhibitors were removed OEGA and AA by purification through a basic and neutral aluminum oxide (Al₂O₃) columns for OEGA and AA, respectively. APS was recrystallized from ethanol/water (1:1) while MBAm was recrystallized from acetone. NIPAM was recrystallized from hexane mixed with a few drops of acetone. All other chemicals were used as received and Milli-Q ultrapure water (18 MΩ H₂O) was used in all procedures unless specified.

Preparation of P(OEGA-co-AA-co-NIPAM) Hydrogels.

P(OEGA-co-AA-co-NIPAM) copolymers were prepared by a free radical polymerization method. In a typical synthesis, 1 g of monomers (OEGA, AA, and NIPAM at various mass ratios) and 0.1 mmol of MBAm was dissolved in 10 mL of water and purged under N₂ for 5 min. The mixture was then transferred to a 25-mL stopcock, air free, Schlenk flask protected with N₂. Three freeze-pump-thaw cycles with Ar were performed to remove dissolved oxygen from the reaction mixture before it was incubated at 70° C. under Ar. After 30 min, 200 mL of aqueous APS (15 mg) was added to the flask under Ar flow, followed by another three freeze-pump-thaw cycles. The reaction was left to run for 5 min at 70° C. under magnetic stirring at 350 rpm. After another 5 min, the reaction was quenched by cooling the flask in an ice bath for 15 min. The mixture was then dialyzed in MWCO 2 kDa membrane in 1 L of water for a week while refreshing water every day to remove monomer, crosslinker, and initiator residues. The resulting product was lyophilized under vacuum to give a copolymer powder. Each copolymer was swollen in 10 mL of 1×DPBS buffer for 24 h to form a hydrogel.

Characterization Methods.

The vibrational spectra of the copolymers were recorded between 400-4000 cm⁻¹ at a resolution of 4 cm⁻¹ on Fourier-transform infrared (FTIR) spectrometer (Shimadzu IRAffinity-1S). The samples were prepared by a KBr pellet method using ˜10 mg of each lyophilized copolymer. Thermal properties of the copolymers were investigated using differential scanning calorimeter (DSC, Shimadzu DSC-60). Each copolymer (5 mg) was put in a standard aluminum sample pan and covered with aluminum lid. The pan was crimped with a crimp press SSCP-1 for the DSC measurement with an empty aluminum sample pan as a control. Measurements were carried out at the rate of 10° C./min from 50-500° C. The thermograms were taken using a TGA instrument (TA Q50) where 25 mg of each hydrogel was used. The sample was equilibrated for 10 min at 30° C. and run under N₂ at a flow rate of 60 mL/min from 30 to 500° C. at a rate of 10° C./min.

Cytotoxicity Studies.

Cytotoxicity studies of P(OEGA-co-AA-co-NIPAM) were performed. In a typical procedure, polymer was lyophilized for 3 days. Around 200 mg of the lyophilized powder was sterilized under UV light for 2 h. Each of the copolymers tested was mixed in DMEM culture media for 72 h at 37° C. in CO₂ incubator (5%). Fibroblast (NIH3T3) cells were then incubated in culture media to obtain 80% confluency. The copolymers were isolated from the culture media (stock: 0.001 g/μL) and diluted with fresh media to obtain different concentrations ranging from 0-30% polymer. Polymers at different concentrations (mg/mL) were added to the 96-well plate coated with cells to a total volume of 100 μl and cell number of 10,000 cells/well. After 72 h incubation, the viability of the fibroblast cells was measured by MTT assay. The viability of cells grown in the media without polymer treatment was used as a control.

Proteins Release Studies.

Release studies of the injectable P(OEGA-co-AA-co-NIPAM) hydrogel were conducted in 12 mm transwell membrane plates. In a typical experiment, around 200 μL of the injectable hydrogel was mixed with a desired amount of protein at the final concentration of 1 mg/mL and the mix was stored at 4° C. overnight. After incubation, the mixture was transferred to a transwell membrane insert (12 mm diameter, 3 μm pore size) then immersed in 1.5 mL of DPBS (pH 7.4). The sample was shaken at 150 rpm throughout the release experiment at 37° C. At different time points, 1.5 mL medium in each well containing the released protein was taken for fluorescence measurement while each well was replaced by fresh medium. The emission spectra were collected at emission wavelengths of 308 nm (with λₑₓ=280 nm) and 345 nm (with λₑₓ=295 nm) for hFGF1 and BSA, respectively. Percentage of cumulative released proteins was calculated based on the calibration curve corresponding to each protein. After fluorescence measurements, the samples were kept at 4° C. for bioactivity assays if applicable. The bioactivity of released hFGF1 was evaluated by cell proliferation assay. Briefly, NIH3T3 cells were grown to 80% confluency and then incubated with 50 ng/mL of the released protein/hFGF1 in serum-supplemented media at a concentration of 10,000 cell per well. The cell viability was determined by Cell Tilter-Glo luminescent assay.

Animal Study for Wound Closure.

C57BL/6J mice (12 weeks old, male, n=11) were anesthetized with 4% isoflurane gas and maintained at 2% isoflurane gas. Each mouse received a subcutaneous injection of an analgesic (Carprofen, 5 mg/kg) before creating a 6 mm full-thickness, excisional wound on the dorsum using a sterile biopsy punch. For each mouse, 100 L of either an hFGF-loaded gel (n=6 mice) or an unloaded control gel (n=5 mice) was applied to each wound. Sterile hydrogels were used to load hFGF at a final concentration of 1 mg/mL. Borax (0.1 w/v %) was used as antiseptic in the PBS for in vivo experiment. Borax (0.1 w/v %) was used as antiseptic in the PBS for in vivo experiment. Wounds were bandaged with Tegaderm and a secondary layer of surgical tape to prevent Tegaderm removal. Wound size was monitored by tracing the wound borders over acetate paper on days 0, 1, 3, 5, 7, and 10. The wound tracings were digitized and average wound area was calculated using Custom MATLAB code that quantified the area within each trace. Wound size measurements were normalized relative to the initial wound size at Day 0.

Results and Discussion

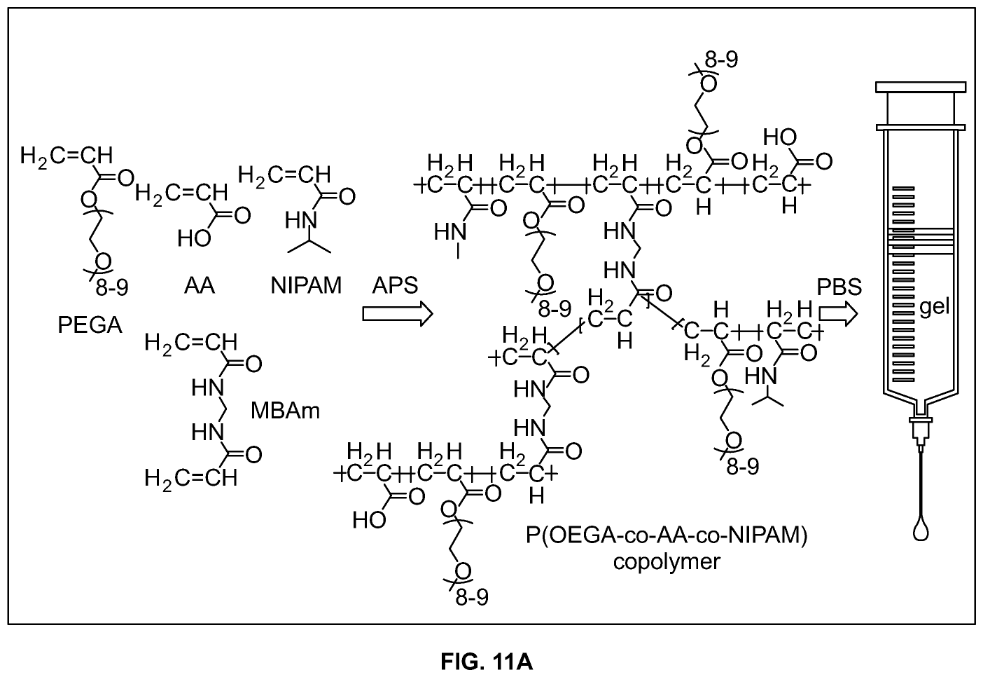

Different formulations of P(OEGA-co-AA-co-NIPAM) were synthesized by a free radical polymerization reaction with modifications to copolymerize with NIPAM. FIG. 11A displays the reaction scheme of the copolymerization of three monomers, OEGA, AA and NIPAM in aqueous medium to form P(OEGA-co-AA-co-NIPAM) with APS as an initiator and MBAm as a crosslinker. The copolymer was then swelled in buffer solution to form hydrogel. Eight different formulations were prepared by systematically changing mass ratios among the three monomers to evaluate the effect(s) of each monomer on copolymer properties. Table 4 lists the reaction parameters and conditions. After the synthesis, the copolymers were swelled in aqueous solution to become hydrogels. The viscosity of the gels was qualitatively visualized by their relative fluidity. The fluidity of the P(OEGA-co-AA-co-NIPAM) hydrogels increases with the increased molar percent of OEGA in the formulation. The order of increasing PEOGA molar percent in hydrogel formulations was Gel 1<Gel 3<Gel 8<Gel 4<Gel 7<Gel 5<Gel 6<Gel 2.

As can been seen in FIG. 11B, Gel 1 having the lowest OEGA content was very viscous, and the viscosity decreased in the order of Gel 1>Gel 3>Gel 2. This result is consistent with previous observation for the P(OEGA-co-AA) hydrogels, wherein AA increased viscosity of the hydrogel due to stronger hydrogen bonding and other intermolecular interactions in the copolymer network. Similar to AA, NIPAM also strengthened the intermolecular interactions in the copolymer compared to POEGA alone.

TABLE 4
Synthesis parameters and reaction conditions of different P(OEGA-co-AA-co-NIPAM)
copolymers to form injectable anionic hydrogel formulations.

| | | OEGA/AA/ | | MBAm/ | | | | |
|---|---|---|---|---|---|---|---|---|
| | OEGA/AA/ | NIPAM | | (OEGA+AA+ | | | | |
| Sample | NIPAM | (at/at/at, | MBAM | NIPAM) | APS | Time | Temp. | Volume |
| ID | (w/w/w, mg) | mmol) | (mmol) | (at/at, mmol) | (mg) | (min) | (° C.) | (mL) |
| Gel 1 | 700/200/100 | 1.46/2.78/0.88 | 0.1 | 0.1/5.12 | 15 | 5 | 70 | 10 |
| Gel 2 | 900/50/50 | 1.88/0.14/0.80 | 0.1 | 0.1/2.81 | 15 | 5 | 70 | 10 |
| Gel 3 | 900/75/25 | 1.67/2.08/0.44 | 0.1 | 0.1/4.19 | 15 | 5 | 70 | 10 |
| Gel 4 | 850/100/50 | 1.77/1.39/0.44 | 0.1 | 0.1/3.60 | 15 | 5 | 70 | 10 |
| Gel 5 | 800/100/100 | 1.88/1.04/0.22 | 0.1 | 0.1/3.14 | 15 | 5 | 70 | 10 |
| Gel 6 | 800/150/50 | 1.88/0.69/0.44 | 0.1 | 0.1/3.01 | 15 | 5 | 70 | 10 |
| Gel 7 | 900/10/90 | 1.77/0.35/1.10 | 0.1 | 0.1/3.22 | 15 | 5 | 70 | 10 |
| Gel 8 | 850/25/125 | 1.67/1.39/0.88 | 0.1 | 0.1/3.94 | 15 | 5 | 70 | 10 |

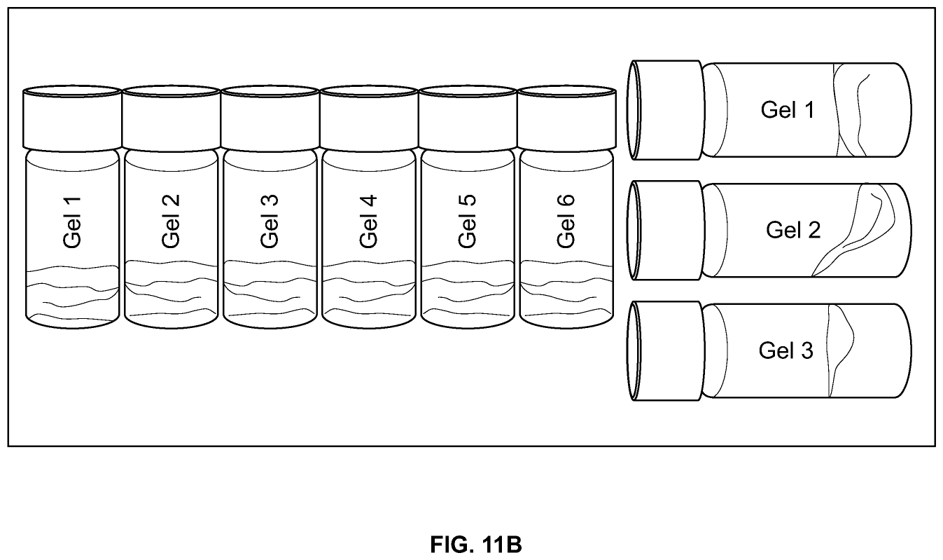

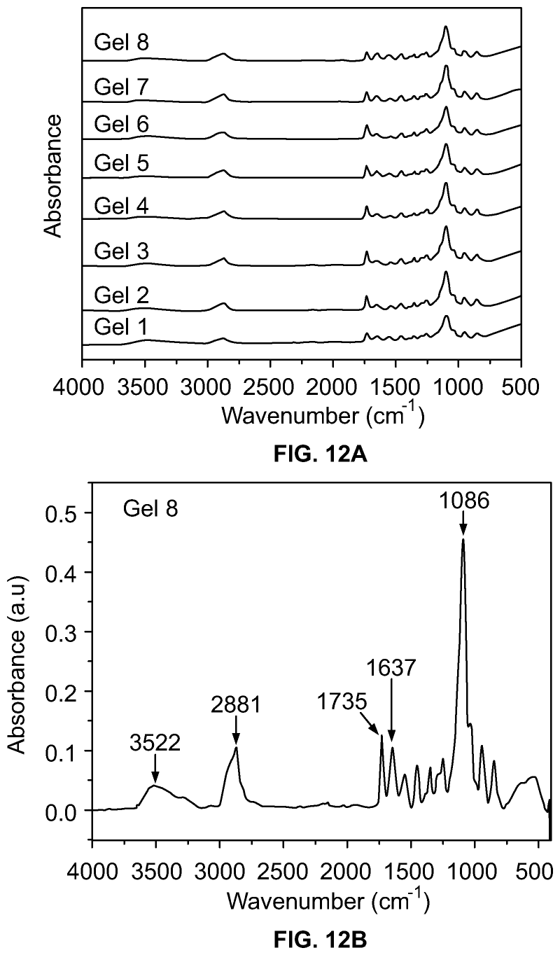

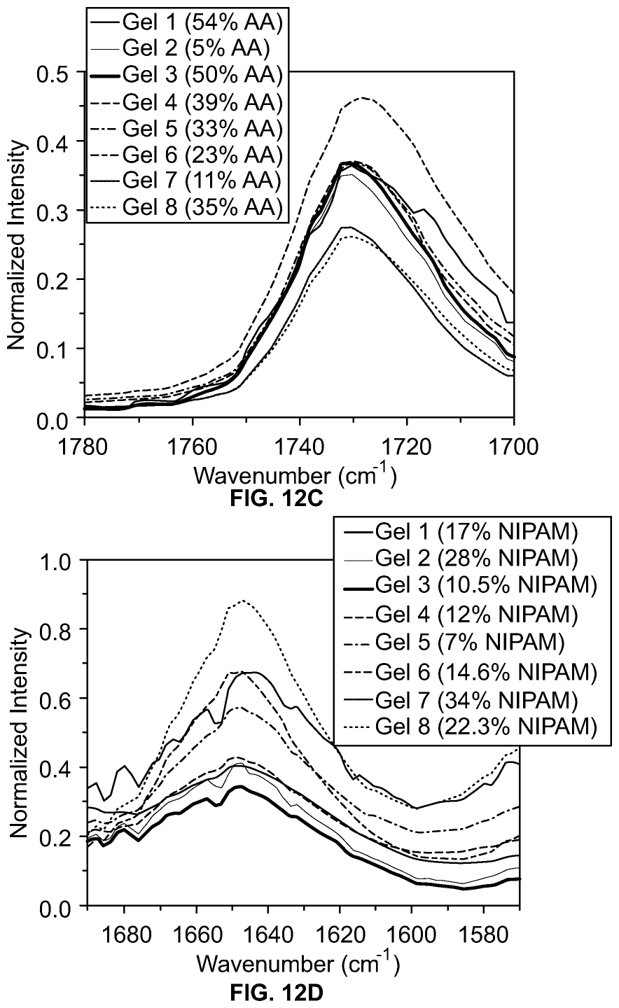

The copolymers were characterized by FTIR spectra to confirm that their chemical structures contained the functional groups from the monomers. FIG. 12A displays the FTIR spectra of all the copolymers which exhibit similar major peaks representing functional groups of the monomers. The spectrum of P(OEGA-co-AA-co-NIPAM) copolymer for Gel 8 was zoomed in to interpret the peaks observed in FIG. 12B. The broad band around 3500 cm⁻¹ can be assigned to N—H stretching from PNIPAM and MBAm. The peak at 2870 cm⁻¹ can be assigned to C—H of the copolymer with a shoulder close to 3000 cm⁻¹ assigned to O—H stretching from PAA component. The peaks at 1730 cm⁻¹ and 1645 cm⁻¹ can be assigned to the carbonyl C═O stretching for ester/carbonate (O═C—O) and amide (O═C—N), respectively. The peak at 1095 cm⁻¹ can be assigned to C—O from POEGA. FIG. 12C shows the ester/carbonate C═O peak intensity (1730 cm⁻¹) normalized to that of the C—O at 1095 cm⁻¹. Likewise, FIG. 12D depicts the C═O peak intensity at 1645 cm⁻¹ for amide normalized to ester/carbonate C═O peak intensity at 1730 cm⁻¹. The increase in the peak intensity at 1645 cm⁻¹ is in accordance with the increase of crosslinking density which is on order of (Gel 8>Gel 1>Gel 7>Gel 5>Gel 4>Gel 6>Gel 2>Gel 3 as it can be seen in Table 4. This can be explained by the fact that more crosslinking density between all monomers will lead to high crosslinker (MBAm) to monomers ratio, thus amide groups in the polymer.

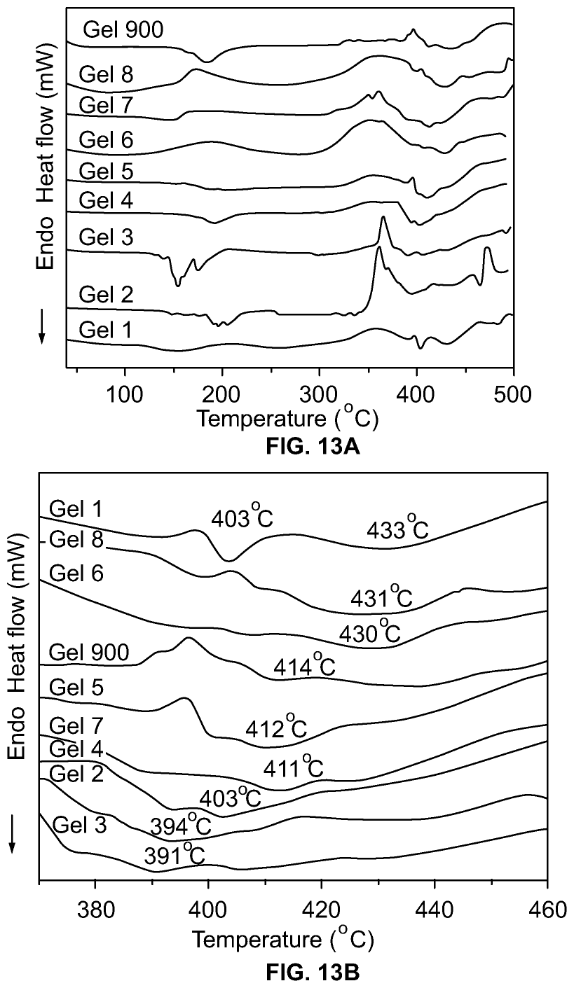

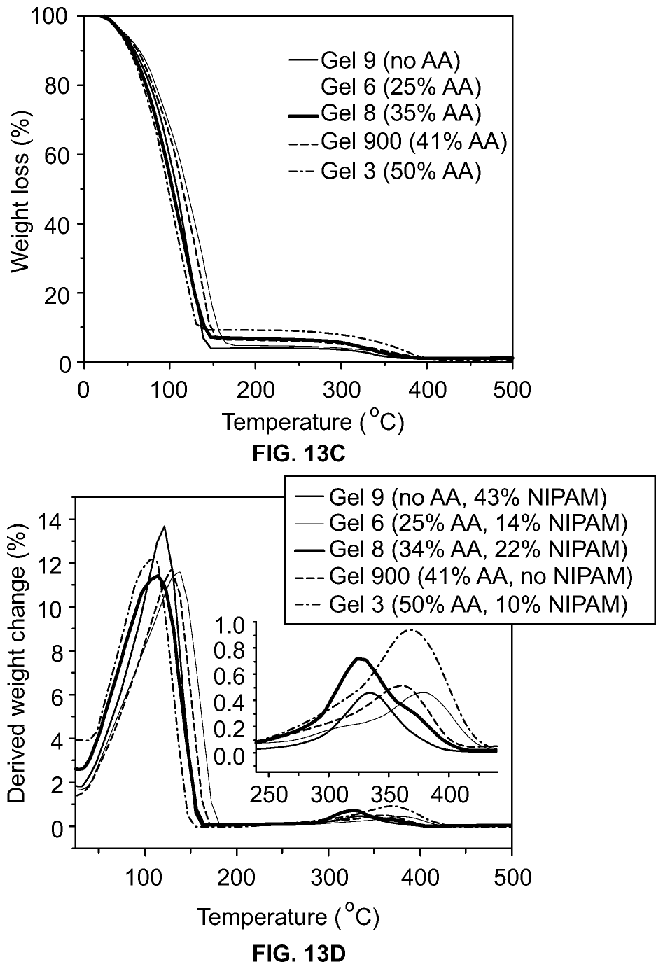

Differential scanning calorimetry (DSC) and thermogravimetric analysis (TGA) were used to study the thermal properties of the copolymers based on P(OEGA-co-AA-co-NIPAM) and Gel 9 00 (POEGA-co-AA) was added for comparison. DSC thermograms show different behaviors of the copolymers, but mostly similar behaviors were observed between 25-100° C. (FIG. 13A). It is believed that the peaks observed around 100° C. for all the copolymers is the evaporation of water in the copolymers before melting point is reached (endothermic process) at higher temperature. The endothermic peaks were observed between 3900-450° C. range following exothermic peaks around 350° C. for all measured copolymers (FIGS. 13A and 13B). After 350° C., the endothermic process could be an indication of the beginning of melting of some of the monomers. Gel 1, having the highest percentage of AA (54%) of all formulations showed the highest melting point at 433° C. (FIG. 13B). The DSC data suggest that copolymers based on P(OEGA-co-AA-co-NIPAM) melt between 390-430° C. and the melting point is dependent on AA and NIPAM content in the formulation wherein copolymers with low molar percentage of either AA or NIPAM show lower melting temperature. Gel 2 (5% AA) and Gel 3(10% NIPAM) showed the low endothermic peaks at 391 and 394° C., respectively (FIG. 13B) AA give a more stable copolymer because of more hydrogen bonding in the hydrogel network. It is believed that the interactions between NIPAM's hydrophilic groups and AA hydrophilic groups influence thermal stability, so copolymers with more NIPAM and less AA contents like Gel 2 (5% AA and 28% NIPAM) and Gel 7 (11% AA and 34% NIPAM) are instable due to less hydrophilic interactions, therefore, less energy is needed to break the hydrogen bonding causing the phase transition to happen at lower temperature. It is also noted that P(OEGA-co-AA-co-NIPAM) copolymer is stable at the range of temperature where it is intended to be used (physiological conditions) during wound healing. TGA of selected copolymers was performed using a TGA instrument (TA Q50) and TGA curves of P(OEGA-co-AA-co-NIPAM) hydrogels of different molar ratios are presented in FIG. 13C. The peak temperature of water loss was slightly different for each of the hydrogel formulations. The temperature values were obtained from the first derivative plots of the TGA as 112° C. for Gel 9, 108° C. for Gel 3, 115° C. for Gel 8, 130° C. for Gel 900 and 137° C. for GEL 6 (FIG. 13D). As the concentration of AA increased in copolymers, the water evaporation temperature slightly increased (Gel 6 has more AA than Gel 3 and Gel 8) and its water evaporates at higher temperature that the other two formulations. This is due to more hydrogen bonds in the polymer with higher AA content. Further increase in temperature caused the polymers to decompose between 350-380° C. The thermal response of the copolymer is in good agreement with the previous studies on polyvinyl alcohol-copoly (methacrylic acid) hydrogels, suggesting that the copolymers can serve as better carriers for drug delivery. It is worthy to mention that the hydrogel with highest NIPAM content (Gel 9, 43% NIPAM) decomposed at lowest temperature at 323° C. with great mass loss (around 14%) compared to when there is no NIPAM in the hydrogel (Gel 900, 0% NIPAM) or less NIPAM (Gel 3, 10% NIPAM) (FIG. 13D). In addition, high NIPAM content resulted in fast water evaporation and highest decomposition rate at later temperature. The behavior of Gel 9 can be explained by the phase separation at the temperature above PNIPAM′ LCST where it expels water molecules out from the polymer networks due to dominant hydrophobic interactions between its isopropyl groups. Both DSC and TGA suggested that the AA content in the polymer has more effect on thermal response of P(OEGA-co-AA-co-NIPAM) hydrogel linking it to its stability of hydrogel at high temperature.

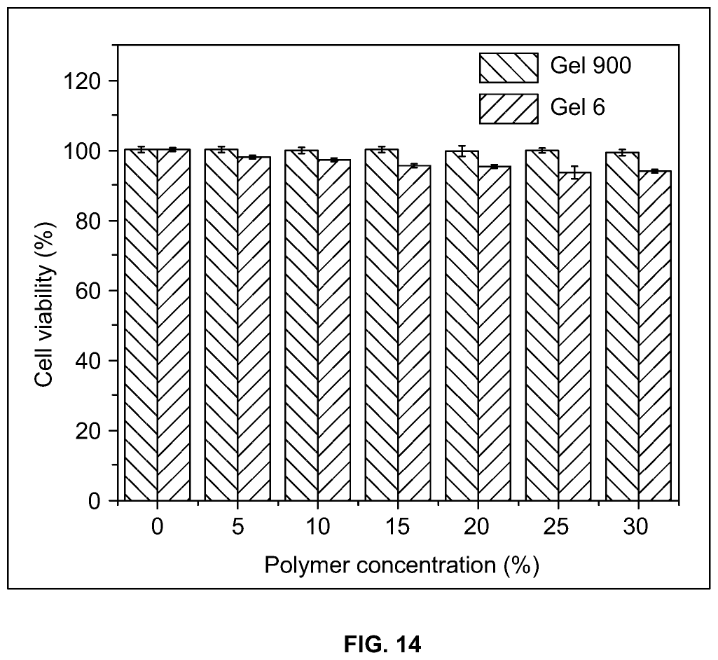

Cytotoxicity assays are used to evaluate biocompatibility of materials intended to be used in biomedical applications. The cytotoxicity of the P(OEGA-co-AA-co-NIPAM) copolymer was evaluated at different polymer concentrations. Fibroblast cells were cultured and exposed to copolymers of different concentrations ranging from 0 to 30 mg/mL of NIPAM. NIPAM concentration was considered because NIPAM monomer has been reported by some studies to have toxicity towards some cell lines. Since the biocompatibility of PNIPAM in hydrogels or nanoparticles is still ambiguous because of contradictory results presented throughout the literature, it is necessary to check cytotoxicity of the PNIPAM components to specific cell lines for the applications. MTT assay data show that the (PEG-PAA-PNIPAM) formulation is not toxic to fibroblast cells at the highest concentration used (30 mg/ml). FIG. 14 indicates that the cell viability is nearly 100% and it is comparable to the control (Gel 900) that was previously proven to be biocompatible. It is also believed that when cell viability is above 70% after being exposed to a compound, then that compound is not cytotoxic. This agrees with other studies which found that concentration of PNIPAM between 0.01 to 10 mg/mL did not cause any cytotoxicity where cell viability exceeded 100% in some cases. These results suggest that little cytotoxic effect was found for the copolymers after purification to remove the monomer residues.

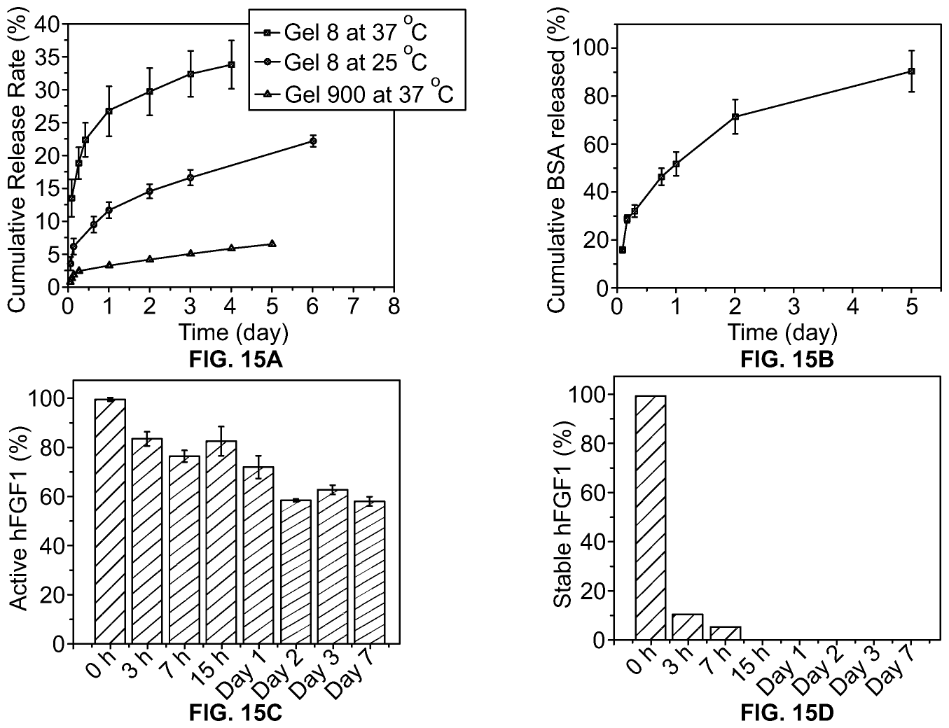

Different formulations of P(OEGA-co-AA-co-NIPAM) hydrogel were tested for their capability for controlled and sustained release of hFGF1 in physiological conditions. Released hFGF1 was monitored by fluorescence spectroscopy for at least 4 days. The released amount of hFGF1 was calculated using a calibration curve and cumulative concentration was obtained. The order of decreasing release is Gel 1>Gel 8>Gel 7>Gel 6>Gel 5>Gel 3>Gel 4>Gel 2. Gel 1 showed the fastest release rate because of the lowest crosslinking density that was easier for proteins to diffuse out of the hydrogel matrix; but the gel was very viscous which makes it hardly injectable. Gel 8 was then chosen as the best formulation in this series of P(OEGA-co-AA-co-NIPAM) study to be further investigated for the controlled release mechanism of proteins and compare with our Gel 900 developed in the previous study. For P(OEGA-co-AA-co-NIPAM), there was a significant difference between release at room temperature (25° C.) versus at 37° C. (FIG. 15A). The release rate was two-fold slower at 25° C. compared to 37° C. This could be explained by the properties of PNIPAM which collapses at temperature higher than its LCST to facilitate release of loaded protein. Compared to Gel 900-P(OEGA-co-AA), incorporating NIPAM in P(OEGA-co-AA) improved the released profile of hFGF1 by 6 times from being less than 7% to 45% in 5 days in physiological conditions (FIG. 15A). The result suggests that PNIPAM in the copolymer reduces the global and local charge density and thus weakens the electrostatic interaction between the protein and the hydrogel to facilitate the release. The electrostatic controlled release mechanism was also evident by a much faster release of BSA (nearly 100% release in 5 days) which is a negatively charged protein (FIG. 15B).

Following the release study, we evaluated the bioactivity of the released hFGF1. The result from the cell proliferation assay indicated that the released hFGF1 remained 60% active even after 7 days in the hydrogels (FIG. 15C). The protein activity of the released hFGF1 declined overtime because of some denaturation and/or aggregations. Stability of hFGF1 in culture media was also evaluated. hFGF1 is known to be unstable and prone to degradation by proteolytic enzymes in DMEM cell culture media at 37° C. FIG. 15D shows that hFGF1 was completely degraded at 37° C. after 24 hours of incubation in the cell culture medium. hFGF1 is unstable and its instability can limit its applications in physiological conditions. The hFGF1 requires binding to heparin to protect it against denaturation and increase its in vivo half-life. However, heparin does not guarantee stability of hFGF1 in some cases. For instance, hFGF1 may lose its activity after 6 hours at 37° C. even in presence of heparin. As an alternative, hFGF1 can be mutated to increase its stability. It is believed that the sustained release of hFGF1 from P(OEGA-co-AA-co-NIPAM) was made possible by strong electrostatic interactions between the anionic hydrogel and positively charged proteins in physiological conditions. These interactions are thought to stabilize the proteins in the hydrogel network to maintain their biological activity over a period of at least ten days before it starts getting inactive. The sustained bioactivity of other growth factors upon encapsulation and release from hydrogels have been reported in literature where released growth factors enable higher cells' differentiation and proliferation compared to growth factors alone.

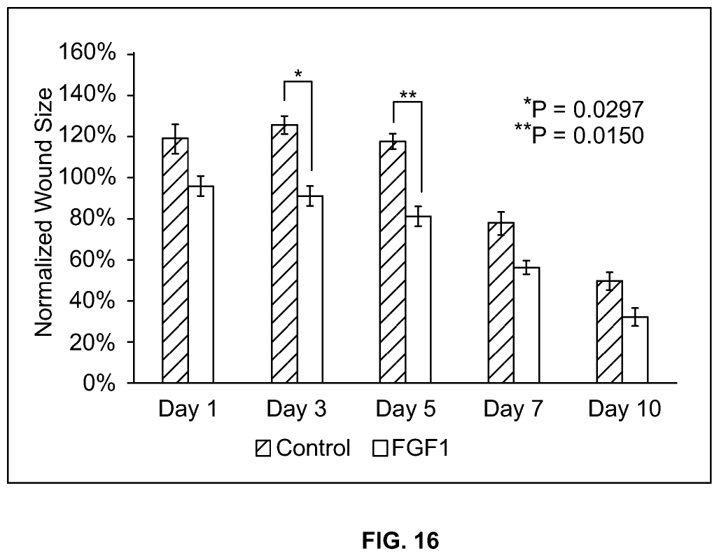

An in vivo study was conducted to evaluate the controlled release of hFGF1 from P(OEGA-co-AA-co-NIPAM) hydrogel (Gel 8) on wound healing. Wound closure data showed that hFGF1 treatment significantly diminished the average wound size (p=0.0047) over the whole data set (FIG. 16). The hFGF1-treated wounds were significantly smaller than the untreated control group on days 3 (p=0.0297) and 5 (p=0.015), which indicate that the effects of hFGF1 were most obvious during the intermediate time points during the proliferative phases of healing (FIG. 16). The significant difference in wound closure is an indication of preserved bioactivity of released hFGF1 that was observed in vitro. The hydrogel was able to prolong the therapeutic effect of loaded hFGF1 to enhance wound healing process compared to control.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiments, methods, and examples, but by all embodiments and methods within the scope and spirit of the disclosure.

1. A method of producing an anionic hydrogel comprising the steps of: providing a) poly(oligoethylene glycol monoacrylate), acrylic acid and N,N′-methylenebis(acrylamide), and water in a container bubbled with a gas before sealing said container; b) incubating; c) adding an initiator; d) performing one or more evacuate-refill cycles; e) terminating the polymerization by cooling; f) dialyzing against water using dialysis membrane; g) lyophilizing into a form of dry gel; and h) adding a wild-type fibroblast growth factor 1 (wtFGF1) and swelling said gel to form an anionic hydrogel.

2. The method of claim 1 further including the step of adding a neutral species to said poly(oligoethylene glycol monoacrylate) and said acrylic acid, said neutral species spaces out the charge distribution of said anionic hydrogel allowing for the increased release of said a wild-type fibroblast growth factor 1 (wtFGF1).

3. The method of claim 2 wherein said neutral species is NIPAM.

4. The method of claim 3 wherein the weight ratio of said anionic hydrogel is poly(oligoethylene glycol monoacrylate) 850, acrylic acid 25, NIPAM 125.

5. The method of claim 2 wherein said neutral species is gelatin.

6. The method of claim 3 wherein the weight ratio of said anionic hydrogel is poly(oligoethylene glycol monoacrylate) 850, acrylic acid 25, gelatin 160.

7. An anionic hydrogel for wound healing comprising: poly(oligoethylene glycol monoacrylate), acrylic, a neutral species and a wild-type fibroblast growth factor 1 (wtFGF1).

8. The anionic hydrogel of claim 7 wherein said neutral species is NIPAM.

9. The anionic hydrogel of claim 8 wherein the weight ratio of said anionic hydrogel is poly(oligoethylene glycol monoacrylate) 850, acrylic acid 25, NIPAM 125.

10. The anionic hydrogel of claim 7 wherein said neutral species is gelatin.

11. The anionic hydrogel of claim 10 wherein the weight ratio of said anionic hydrogel is poly(oligoethylene glycol monoacrylate) 850, acrylic acid 25, gelatin 125.

12. A method of to enhance cell growth and cell adhesion to treat a wound in a mammal comprising the steps:
applying an anionic hydrogel to the wound;
said anionic hydrogel comprising: poly(oligoethylene glycol monoacrylate), acrylic, a neutral species and a wild-type fibroblast growth factor 1 (wtFGF1); and
said hydrogel configured to controllably release said a wild-type fibroblast growth factor 1 (wtFGF1).

13. The method of claim 12 wherein said hydrogel inhibits the degradation of said a wild-type fibroblast growth factor 1 (wtFGF1).

14. The method of claim 13 further including the step of increasing said neutral species to said poly(oligoethylene glycol monoacrylate) and said acrylic acid, to increase the release of said a wild-type fibroblast growth factor 1 (wtFGF1).

15. The method of claim 14 wherein said neutral species is NIPAM.

16. The method of claim 14 wherein the weight ratio of said anionic hydrogel is poly(oligoethylene glycol monoacrylate) 850, acrylic acid 25, NIPAM 125.

17. The anionic hydrogel of claim 14 wherein said neutral species is gelatin.

18. The anionic hydrogel of claim 7 wherein said neutral species is NIPAM.

19. The anionic hydrogel of claim 8 wherein the weight ratio of said anionic hydrogel is poly(oligoethylene glycol monoacrylate) 850, acrylic acid 25, NIPAM 125.

20. The anionic hydrogel of claim 7 wherein said neutral species is gelatin.

21. The anionic hydrogel of claim 10 wherein the weight ratio of said anionic hydrogel is poly(oligoethylene glycol monoacrylate) 850, acrylic acid 25, gelatin 160.
